(19) United States
(12) Patent Application Publication
(10) Pub. No.: US 2024/0325612 A1
(43) Pub. Date: Oct. 3, 2024

(54) ACOUSTIC MATCHING MATERIALS AND METHODS FOR PREPARING THEREOF, AND ULTRASONIC PROBES

(71) Applicant: WUHAN UNITED IMAGING HEALTHCARE CO., LTD., Wuhan (CN)

(72) Inventors: Kang SI, Shanghai (CN); Yu LIU, Wuhan (CN); Zhifang LI, Wuhan (CN); Cong LIU, Wuhan (CN)

(73) Assignee: WUHAN UNITED IMAGING HEALTHCARE CO., LTD., Wuhan (CN)

(21) Appl. No.: 18/623,005

(22) Filed: Mar. 31, 2024

(30) Foreign Application Priority Data
Mar. 31, 2023 (CN) 202310340432.6

(51) Int. Cl. A61L 31/12; A61B 8/00

(52) U.S. Cl. CPC A61L 31/129; A61B 8/445; A61L 2300/802

(57) ABSTRACT

The present disclosure provides an acoustic matching material applied to a matching layer of an ultrasonic probe. The acoustic matching material is prepared by adding additive into a matrix to form a mixture and curing the mixture. The matrix includes vinyl ester resin and a curing agent. The additive includes at least one of a thixotropic agent, a thickener, or a modified filler.

### CROSS REFERENCE TO RELATED APPLICATIONS

This application claims priority to Chinese Patent Application No. 202310340432.6 filed on Mar. 31, 2023, the entire contents of which are hereby incorporated by reference.

### TECHNICAL FIELD

The present disclosure relates to the technical field of acoustic materials, and in particular, to acoustic matching materials and methods for preparing thereof, and ultrasonic probes using the acoustic matching materials.

### BACKGROUND

Ultrasonic imaging, as a non-invasive, ionizing radiation-free, and easy-to-operate imaging method, is widely used in clinical diagnosis. The principle of ultrasonic imaging is to use electrical signals to excite a piezoelectric element in an ultrasonic probe to generate ultrasonic waves, which propagate through a matching layer in the ultrasonic probe and enter the human body. Different tissues or organs in the human body have different reflection coefficients of ultrasonic waves. The ultrasonic probe receives reflected signals of the ultrasonic waves from different tissues or organs in the human body and converts the reflected signals into electrical signals. The converted electrical signals are then processed to generate ultrasonic images. The matching layer is an important component of the ultrasonic probe, and the matching layer's acoustic properties, such as sound attenuation, acoustic impedance, etc., will affect the performance of the ultrasonic probe and the quality of ultrasonic imaging. Therefore, it is desirable to provide high performance acoustic matching material applied to the matching layer of the ultrasonic probe.

### SUMMARY

An aspect of the present disclosure relates to an acoustic matching material applied to a matching layer of an ultrasonic probe. The acoustic matching material is prepared by adding additive into a matrix to form a mixture and curing the mixture. The matrix includes vinyl ester resin and a curing agent. The additive includes at least one of a thixotropic agent, a thickener, or a modified filler.

In some embodiments, at a frequency of 5 MHz, a sound attenuation of the acoustic matching material is less than 27 dB/cm.

In some embodiments, at the frequency of 5 MHz, the sound attenuation of the acoustic matching material is less than 16 dB/cm.

In some embodiments, at the frequency of 5 MHz, the sound attenuation of the acoustic matching material is less than 7 dB/cm.

In some embodiments, a mass fraction of the vinyl ester resin is 100 parts, a mass fraction of the curing agent is 1-2 parts, a mass fraction of the thixotropic agent is 0-0.5 parts, a mass fraction of the thickener is 0-4 parts, and/or a mass fraction of the modified filler is 0-400 parts.

In some embodiments, a volume of the modified filler is 0-50% of a total volume of the mixture.

In some embodiments, the vinyl ester resin includes at least one of bisphenol A epoxy vinyl ester resin, phenolic epoxy vinyl ester resin, or addition polymerization epoxy vinyl ester resin with main chain structure, and the curing agent includes methyl ethyl ketone peroxide.

In some embodiments, the thixotropic agent includes at least one of hydrogenated castor oil or polyamide wax.

In some embodiments, the thickener includes at least one of bentonite or fumed silica.

In some embodiments, the modified filler is prepared by performing surface activation modification on a filler. The filler includes at least one of hollow glass microsphere, glass microsphere, aluminum oxide microsphere, aluminum oxide powder, tungsten powder, tungsten trioxide powder, copper powder, iron powder, iron oxide powder, rubber powder, or plastic powder.

In some embodiments, the modified filler is prepared by preparing a processed filler by soaking and stirring the filler in alcohol and a coupling agent, the coupling agent includes at least one of a silane coupling agent or a titanate coupling agent; and preparing the modified filler by drying the processed filler.

In some embodiments, acoustic matching materials with different acoustic impedances are prepared by changing a type and an addition ratio of the additive.

Another aspect of the present disclosure relates to a method for preparing an acoustic matching material applied to a matching layer of an ultrasonic probe. The method includes adding additive into a matrix to form a mixture, the matrix including vinyl ester resin and a curing agent, the additive including at least one of a thixotropic agent, a thickener, or a modified filler; and curing the mixture.

A still further aspect of the present disclosure relates to an ultrasonic probe, comprising a matching layer that is made of an acoustic matching material. The acoustic matching material is prepared by adding additive into a matrix to form a mixture, the matrix including vinyl ester resin and a curing agent, the additive including at least one of a thixotropic agent, a thickener, or a modified filler; and curing the mixture.

In some embodiments, different matching layers of the ultrasonic probe are made of acoustic matching materials with different acoustic impedances.

Additional features may be set forth in part in the description which follows, and in part may become apparent to those skilled in the art upon examination of the following and the accompanying drawings or may be learned by production or operation of the examples. of the present disclosure may be realized and attained by practice or use of various aspects of the methodologies, instrumentalities, materials, experiment procedures, compounds, and combinations set forth in the detailed examples discussed below.

### BRIEF DESCRIPTION OF THE DRAWINGS

The present disclosure is further described in terms of example embodiments. These example embodiments are described in detail with reference to the drawings. These embodiments are non-limiting example embodiments, in which like reference numerals represent similar structures throughout the several views of the drawings, and wherein:

FIG. 1 is a schematic structural diagram illustrating an exemplary ultrasonic imaging device according to some embodiments of the present disclosure; and

FIG. 2 is a flowchart illustrating an exemplary method for preparing an acoustic matching material according to some embodiments of the present disclosure.

### DETAILED DESCRIPTION

In order to illustrate the technical solutions related to the embodiments of the present disclosure, a brief introduction of the drawings referred to in the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless stated otherwise or obvious from the context, the same reference numeral in the drawings refers to the same structure and operation.

As used in the disclosure and the appended claims, the singular forms “a,” “an,” and “the” include plural referents unless the content clearly dictates otherwise. In general, the terms “comprise” and “include” merely prompt to include steps and elements that have been clearly identified, and these steps and elements do not constitute an exclusive listing. The methods or the devices may also include other steps or elements.

Numerical ranges used herein are intended to concisely and concisely describe each value included within that range.

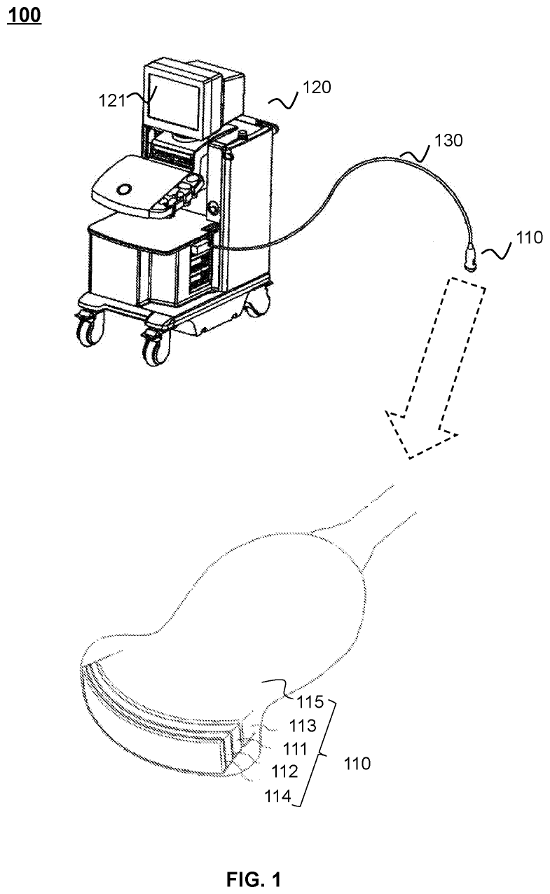

FIG. 1 is a schematic structural diagram illustrating an exemplary ultrasonic imaging device 100 according to some embodiments of the present disclosure. As shown in FIG. 1, the ultrasonic imaging device 100 may include an ultrasonic probe 110 and a device host 120.

The ultrasonic probe 110 may transmit and receive ultrasonic waves, and perform an electro-acoustic signal converting process of the ultrasonic waves. The ultrasonic probe 110 may convert a first electrical signal transmitted by the device host 120 into a high-frequency oscillating ultrasonic signal, and may also convert the reflected ultrasonic signal from an imaging object, such as an organ or tissue, into a second electrical signal.

The device host 120 may be configured to process the second electrical signal received by the ultrasonic probe 110 to generate an ultrasonic image of the imaging object.

In some embodiments, the device host 120 includes a central processing unit (CPU), an application-specific integrated circuit (ASIC), an application-specific instruction set processor (ASIP), a graphics processor (GPU), a physical processing unit (PPU), a digital signal processor (DSP), a field programmable gate array (FPGA), a programmable logic device (PLD), a controller, a microcontroller unit, a reduced instruction set computer (RISC), a microprocessor, or the like, or any combination thereof.

The device host 120 may include a display 121 for displaying information (e.g., ultrasonic signal information, ultrasonic image information, etc.) related to the imaging object. In some embodiments, the display includes a liquid crystal display (LCD), a plasma display, a light emitting diode (LED) display, or the like, or any combination thereof.

The ultrasonic imaging device 100 may include a cable 130 for connecting the ultrasonic probe 110 and the device host 120, and the ultrasonic probe 110 and the device host 120 may communicate (e.g., transmit data to each other) through the cable 130.

As shown in the enlarged view of the ultrasonic probe 110 in FIG. 1, the ultrasonic probe 110 may include a piezoelectric element 111, at least one matching layer 112, a backing layer 113, an acoustic lens 114, and a support frame 115.

The piezoelectric element 111 may vibrate to generate ultrasonic waves by applying a voltage to electrodes at both ends of the piezoelectric element 111. The ultrasonic waves emitted from the piezoelectric element 111 may propagate through the at least one matching layer 112 and the acoustic lens 114 in turn, and then focus on the imaging object. The reflected ultrasonic waves from the imaging object may propagate through the acoustic lens 114 and the at least one matching layer 112 in turn, and then converge on the piezoelectric element 111. The reflected ultrasonic waves may carry information (e.g., information about the reflection, absorption, and scattering of ultrasonic waves) about the imaging object. The information may be received by the piezoelectric element 111 and an electro-acoustic signal converting process may be performed on the reflected ultrasonic waves to obtain the second electrical signal carrying the information.

The acoustic lens 114 may be disposed on a surface of the ultrasonic probe 110 and in contact with the imaging object when the ultrasonic probe 110 is configured to scan the imaging object.

The backing layer 113 may be located on a side of the piezoelectric element 111 away from the acoustic lens 114. The backing layer 113 may reduce a pulse width of the ultrasonic waves by suppressing excessive vibration of the piezoelectric element 111, to improve an axial resolution of an ultrasonic image.

The support frame 115 is configured to support and protect the piezoelectric element 111, the backing layer 113, the at least one matching layer 112, and the acoustic lens 114. The backing layer 113, the piezoelectric element 111, and the at least one matching layer 112, and the acoustic lens 114 may be sequentially disposed on the support frame 115.

The at least one matching layer 112 is located on a side of the piezoelectric element 111 close to the acoustic lens 114. The matching layer 112 may be configured to reduce a difference between the acoustic impedance of the piezoelectric element 111 and the acoustic impedance of an object (e.g., a body surface of the imaging object) to realize efficient transmission and reception of the ultrasonic waves.

As mentioned above, the ultrasonic waves generated by the piezoelectric element 111 pass through the at least one matching layer 112 and can further be incident into the imaging object through the acoustic lens 114. The at least one matching layer 112 needs to have low sound attenuation. The sound attenuation of a medium refers to the amount of sound energy attenuation due to sound wave divergence, absorption, reflection, scattering, and other reasons when propagating in the medium. The smaller the sound attenuation, the smaller the sound energy loss of the ultrasonic waves, and the better the imaging effect of the imaging object.

Generally, the material applied to the at least one matching layer 112 of the ultrasonic probe 110 is prepared using epoxy resin as a matrix. However, the epoxy resin has large sound attenuation, therefore, the sound attenuation of the material made of epoxy resin makes it difficult to meet the requirements of the at least one matching layer 112. In order to solve the above problems, the embodiments of the present disclosure provide an acoustic matching material applied to the at least one matching layer 112. In the embodiments of the present disclosure, the acoustic matching material is prepared based on vinyl ester resin (also referred to as epoxy vinyl ester resin). In some embodiments, the acoustic matching material is prepared by curing pure vinyl ester resin. In some embodiments, the acoustic matching material is prepared by curing a matrix that includes vinyl ester resin and a curing agent. The curing agent refers to a substance that is used to promote or control the curing reaction of the vinyl ester resin. For example, after being added to the vinyl ester resin, the curing agent decomposes to form free radicals, which promote the opening of the unsaturated double bonds in the vinyl ester resin for chain growth polymerization. It should be noted that the curing agent only triggers but does not participate in the reaction of the vinyl ester resin.

In some embodiments, the acoustic matching material is prepared by adding additive into the matrix to form a mixture and curing the mixture. The additive may include at least one of a thixotropic agent, a thickener, or a modified filler. The thixotropic agent refers to a substance that uses the thixotropic effect to make a liquid resin have a greater viscosity when it is in a static state, thereby preventing the liquid resin from sagging. Adding the thixotropic agent to the liquid resin may make the liquid resin have a higher consistency when static and turn into a low-viscosity fluid under the action of external force. The thickener refers to a substance that increases the viscosity of the resin, prevents sagging during the preparation process, and imparts certain mechanical properties and storage stability to the resin. The modified filler refers to a substance that improves the properties of the resin. For example, the additive includes the modified filler. As another example, the additive includes the modified filler and the thixotropic agent. As yet another example, the additive includes the modified filler and the thickener. As yet another example, the additive includes the modified filler, the thixotropic agent, and the thickener.

Under a similar preparation condition, compared with the material prepared using the epoxy resin as the matrix, the acoustic matching material prepared in the embodiments of the present disclosure has a lower sound attenuation, thereby being more suitable for the at least one matching layer 112 of the ultrasonic probe 110. In some embodiments, at a frequency of 5 MHz, the sound attenuation of the acoustic matching material prepared in the embodiments of the present disclosure is less than 27 dB/cm. In some embodiments, at the frequency of 5 MHz, the sound attenuation of the acoustic matching material prepared in the embodiments of the present disclosure is less than 26.1 dB/cm. In some embodiments, at the frequency of 5 MHz, the sound attenuation of the acoustic matching material prepared in the embodiments of the present disclosure is less than 24.3 dB/cm. In some embodiments, at the frequency of 5 MHz, the sound attenuation of the acoustic matching material prepared in the embodiments of the present disclosure is less than 24 dB/cm. In some embodiments, at the frequency of 5 MHz, the sound attenuation of the acoustic matching material prepared in the embodiments of the present disclosure is less than 23.8 dB/cm. In some embodiments, at the frequency of 5 MHz, the sound attenuation of the acoustic matching material prepared in the embodiments of the present disclosure is less than 22.5 dB/cm. In some embodiments, at the frequency of 5 MHz, the sound attenuation of the acoustic matching material prepared in the embodiments of the present disclosure is less than 20 dB/cm. In some embodiments, at the frequency of 5 MHz, the sound attenuation of the acoustic matching material prepared in the embodiments of the present disclosure is less than 19.5 dB/cm. In some embodiments, at the frequency of 5 MHz, the sound attenuation of the acoustic matching material prepared in the embodiments of the present disclosure is less than 17.4 dB/cm. In some embodiments, at the frequency of 5 MHz, the sound attenuation of the acoustic matching material prepared in the embodiments of the present disclosure is less than 16 dB/cm. In some embodiments, at the frequency of 5 MHz, the sound attenuation of the acoustic matching material prepared in the embodiments of the present disclosure is less than 13 dB/cm. In some embodiments, at the frequency of 5 MHz, the sound attenuation of the acoustic matching material prepared in the embodiments of the present disclosure is less than 10 dB/cm. In some embodiments, at the frequency of 5 MHz, the sound attenuation of the acoustic matching material prepared in the embodiments of the present disclosure is less than 7 dB/cm. In some embodiments, at the frequency of 5 MHz, the sound attenuation of the acoustic matching material prepared in the embodiments of the present disclosure is less than 6.3 dB/cm.

Under similar preparation condition, compared with the material prepared using the epoxy resin as the matrix, the acoustic matching material prepared in the embodiments of the present disclosure has a larger flexural modulus. The flexural modulus refers to the ability of a material to resist bending deformation within its elastic limit. The higher the flexural modulus of a material, the higher its mechanical strength, and the better its processability. The acoustic matching material with a larger flexural modulus is used to prepare the at least one matching layer 112 of the ultrasonic probe 110, which can improve the production efficiency of the at least one matching layer 112.

In some embodiments, the flexural modulus of the acoustic matching material prepared in the embodiments of the present disclosure is larger than 2 Gpa. In some embodiments, the flexural modulus of the acoustic matching material prepared in the embodiments of the present disclosure is larger than 2.5 Gpa. In some embodiments, the flexural modulus of the acoustic matching material prepared in the embodiments of the present disclosure is larger than 3 Gpa. In some embodiments, the flexural modulus of the acoustic matching material prepared in the embodiments of the present disclosure is larger than 3.15 Gpa. In some embodiments, the flexural modulus of the acoustic matching material prepared in the embodiments of the present disclosure is larger than 3.2 Gpa. In some embodiments, the flexural modulus of the acoustic matching material prepared in the embodiments of the present disclosure is larger than 3.21 Gpa. In some embodiments, the flexural modulus of the acoustic matching material prepared in the embodiments of the present disclosure is larger than 3.5 Gpa. In some embodiments, the flexural modulus of the acoustic matching material prepared in the embodiments of the present disclosure is larger than 3.54 Gpa. In some embodiments, the flexural modulus of the acoustic matching material prepared in the embodiments of the present disclosure is larger than 3.8 Gpa. In some embodiments, the flexural modulus of the acoustic matching material prepared in the embodiments of the present disclosure is larger than 3.82 Gpa.

A curing mechanism of the acoustic matching material prepared in the embodiments of the present disclosure may be a free radical polymerization mechanism. The free radical polymerization mechanism is characterized by fast growth and fast termination. Compared to the ring-opening polymerization reaction mechanism of the epoxy resin, the acoustic matching material prepared in the embodiments of the present disclosure may cure faster. The acoustic matching material with a faster cure speed is used to prepare the at least one matching layer 112 of the ultrasonic probe 110, which can improve the production efficiency of the at least one matching layer 112.

The material used to make the at least one matching layer 112 of the ultrasonic probe 110 needs to be resistant to high temperatures to avoid deformation due to high temperatures during processing (e.g., grinding or polishing). The temperature resistance of the material prepared using the epoxy resin as the matrix is 80° C. The temperature resistance of the acoustic matching material prepared in the embodiments of the present disclosure can reach 120° C. Therefore, compared with the material prepared using the epoxy resin as the matrix, the acoustic matching material prepared in the embodiments of the present disclosure has better processability.

A difference in acoustic impedance between the piezoelectric element 111 and the imaging object is usually large, which would cause the ultrasonic waves propagated from the piezoelectric element 111 to be reflected on a surface of the imaging object, thus causing the incidence efficiency of the ultrasonic waves to the imaging object to decrease. The acoustic impedance refers to the resistance to the propagation of ultrasonic waves in a medium. In order to suppress the reflection of ultrasonic waves and improve the incidence efficiency of ultrasonic waves to the imaging object, the at least one matching layer 112 provided between the piezoelectric element 111 and the acoustic lens 114 needs to have appropriate acoustic impedance. For example, the acoustic impedance of the at least one matching layer 112 is between the acoustic impedance of the piezoelectric element 111 and the acoustic impedance of the imaged object. Merely by way of example, the acoustic impedance of the piezoelectric element 111 is 33 MRayl, and the acoustic impedance of the imaged object is 1.5 MRayl. As another example, the at least one matching layer 112 may include multiple matching layers with different acoustic impedances. The matching layer close to the imaging object has an acoustic impedance that is close to the acoustic impedance of the imaging object. The matching layer close to the piezoelectric element 111 has an acoustic impedance that is close to the acoustic impedance of the piezoelectric element 111.

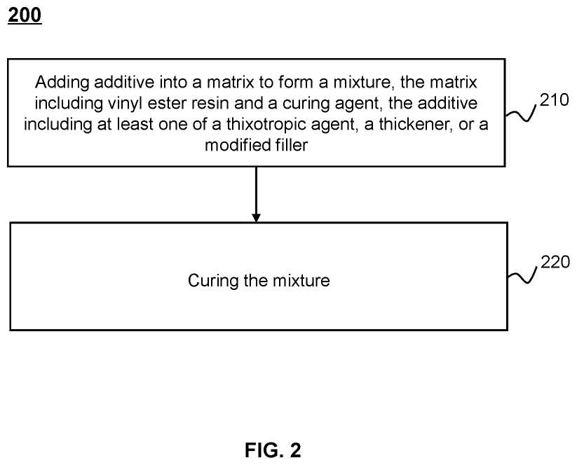

FIG. 2 is a flowchart illustrating an exemplary method for preparing an acoustic matching material according to some embodiments of the present disclosure.

In preparation step 210, additive is added into a matrix to form a mixture. The matrix may include vinyl ester resin and a curing agent, and the additive may include at least one of a thixotropic agent, a thickener, or a modified filler.

In some embodiments, when preparing the acoustic matching material, a mass fraction of the vinyl ester resin is 100 parts. In some embodiments, when preparing the acoustic matching material, a mass fraction of the curing agent is 1-2 parts. For example, the mass fraction of the curing agent is 1-1.5 parts, 1.5-2 parts, 1 part, 1.5 parts, or 2 parts. In some embodiments, when preparing the acoustic matching material, a mass fraction of the thixotropic agent is 0-0.5 parts. For example, the mass fraction of the thixotropic agent is 0-0.4 parts, 0-0.3 parts, 0-0.2 parts, 0-0.1 parts, 0.1-0.5 parts, 0.1-0.4 parts, 0.1-0.3 parts, 0.1-0.2 parts, 0.2-0.5 parts, 0.2-0.4 parts, 0.2-0.3 parts, 0.3-0.5 parts, 0.3-0.5 parts, or 0.4-0.5 parts. In some embodiments, when preparing the acoustic matching material, a mass fraction of the thickener is 0-4 parts. For example, the mass fraction of the thickener is 0-3 parts, 0-2 parts, 0-1 parts, 1-4 parts, 1-3 parts, 1-2 parts, 2-4 parts, 2-3 parts, or 3-4 parts. In some embodiments, when preparing the acoustic matching material, a mass fraction of the modified filler is 0-400 parts. For example, the mass fraction of the modified filler is 0-300 parts, 0-200 parts, 0-100 parts, 100-400 parts, 100-300 parts, 100-200 parts, 200-400 parts, 200-300 parts, or 300-400 parts. In some embodiments, mass fractions of the thixotropic agent, the thickener, and the modified filler are 0 at the same time. In some embodiments, the mass fractions of the thixotropic agent, the thickener, and the modified filler are not 0 at the same time. For example, the mass fraction of the modified filler is not 0, and the mass fractions of the thixotropic agent and/or the thickener are 0. As another example, the mass fractions of the thixotropic agent, the thickener, and the modified filler are not 0.

In some embodiments, when preparing the acoustic matching material, a volume of the modified filler is 0-60% of a total volume of the mixture. For example, the volume of the modified filler is 0-50%, 0-40%, 0-30%, 0-20%, 0-10%, 10-60%, 10-50%, 10-40%, 10-30%, 10-20%, 20-60%, 20-50%, 20-40%, 20-30%, 30-60%, 30-50%, 30-40%, 40-60%, 40-50%, 50-60% of the total volume of the mixture. It should be noted that when the volume of the modified filler is larger than 60% of the total volume of the mixture, the fluidity of the mixture is poor and it cannot be poured and molded.

In some embodiments, when preparing the acoustic matching material, the mass fraction of the vinyl ester resin is 100 parts, and the mass fraction of the curing agent is 2 parts. In some embodiments, when preparing the acoustic matching material, the mass fraction of the thixotropic agent is 0.5 parts, the mass fraction of the thickener is 4 parts, and the mass fraction of the modified filler is 400 parts, or 12 parts, or 150 parts, or 100 parts, or 200 parts, or 30 parts.

In some embodiments, the vinyl ester resin includes at least one of bisphenol A epoxy vinyl ester resin or phenolic epoxy vinyl ester resin. In some embodiments, the vinyl ester resin includes addition polymerization epoxy vinyl ester resin with main chain structure. The main chain structure uses particle vibration to transmit ultrasonic waves through changes in bond length and bond angle, with low energy loss. Therefore, the addition polymerization epoxy vinyl ester resin with main chain structure has the characteristics of low sound attenuation of the addition polymer. Accordingly, the prepared acoustic matching material has low sound attenuation. It should be noted that the vinyl ester resin in the present disclosure is liquid. In some embodiments, the curing agent includes at least one of peroxide or azo compounds. For example, the curing agent includes methyl ethyl ketone peroxide. In some embodiments, the thixotropic agent includes at least one of hydrogenated castor oil or polyamide wax. For example, the thixotropic agent is the hydrogenated castor oil. As another example, the thixotropic agent is the polyamide wax. As yet another example, the thixotropic agent is the hydrogenated castor oil and the polyamide wax. In some embodiments, the thickener includes at least one of bentonite or fumed silica. Merely by way of example, the bentonite is organic bentonite.

In some embodiments, the modified filler is prepared by performing surface activation modification on a filler. The filler may include at least one of hollow glass microsphere, glass microsphere, aluminum oxide microsphere, aluminum oxide powder, tungsten powder, tungsten trioxide powder, copper powder, iron powder, iron oxide powder, rubber powder, or plastic powder. Because a surface area of the filler is relatively large, after the filler is added to the matrix, there would be a very thin air layer between the matrix (e.g., the vinyl ester resin) and the filler, so that the matrix cannot infiltrate the filler, which reduces the performance (e.g., the sound attenuation) of the prepared acoustic matching material. Therefore, the filler needs to be modified, which can improve the compatibility between the matrix and the modified filler so that the modified filler can better combine with the matrix. Further, the filler being modified can adjust the propagation speed of ultrasonic waves in the prepared acoustic matching material, thereby adjusting the acoustic impedance of the prepared acoustic matching material. In addition, the filler being modified can improve the uniformity of the distribution of the modified fillers in the matrix, which allows the prepared acoustic matching material to have uniform performance (e.g. density, sound velocity, sound attenuation, acoustic impedance). As a result, the prepared acoustic matching material has a performance closer to simulation calculations, improving acoustic simulation accuracy. It should be noted that the sound velocity in the present disclosure may refer to the propagation speed of ultrasonic waves.

In some embodiments, when the filler is at least one of the hollow glass microsphere, the glass microsphere, the aluminum oxide microsphere, the aluminum oxide powder, the tungsten powder, the tungsten trioxide powder, the copper powder, the iron powder, the iron oxide powder, the rubber powder, or the plastic powder, the additive includes the thixotropic agent and/or the thickener. In some embodiments, when the filler is the aluminum oxide microsphere and/or the aluminum oxide powder, the additive does not include the thixotropic agent and/or the thickener.

In some embodiments, when the mass of the modified filler is large, the modified filler may precipitate and migrate in the vinyl ester resin, thereby reducing the uniformity of the distribution of the modified filler in the vinyl ester resin. In such cases, it is necessary to add the thixotropic agent, which can reduce the precipitation and migration of the modified filler in the vinyl ester resin, thereby making the modified filler evenly distributed in the vinyl ester resin.

In some embodiments, the vinyl ester resin includes at least one of bisphenol A epoxy vinyl ester resin, phenolic epoxy vinyl ester resin, or addition polymerization epoxy vinyl ester resin with main chain structure, and the curing agent includes methyl ethyl ketone peroxide. In some embodiments, the vinyl ester resin includes at least one of bisphenol A epoxy vinyl ester resin, phenolic epoxy vinyl ester resin, or addition polymerization epoxy vinyl ester resin with main chain structure, the curing agent includes methyl ethyl ketone peroxide, the thixotropic agent is polyamide wax, the thickener is bentonite, and the modified filler is surface activation modified tungsten powder. In some embodiments, the vinyl ester resin includes at least one of bisphenol A epoxy vinyl ester resin, phenolic epoxy vinyl ester resin, or addition polymerization epoxy vinyl ester resin with main chain structure, the curing agent includes methyl ethyl ketone peroxide, the thixotropic agent is polyamide wax, the thickener is bentonite, and the modified filler is surface activation modified hollow glass microsphere. In some embodiments, the vinyl ester resin includes at least one of bisphenol A epoxy vinyl ester resin, phenolic epoxy vinyl ester resin, or addition polymerization epoxy vinyl ester resin with main chain structure, the curing agent includes methyl ethyl ketone peroxide, the thixotropic agent is polyamide wax, the thickener is bentonite, and the modified filler is surface activation modified aluminum oxide microsphere. In some embodiments, the vinyl ester resin includes at least one of bisphenol A epoxy vinyl ester resin, phenolic epoxy vinyl ester resin, or addition polymerization epoxy vinyl ester resin with main chain structure, the curing agent includes methyl ethyl ketone peroxide, the thixotropic agent is polyamide wax, the thickener is bentonite, and the modified filler is surface activation modified glass microsphere. In some embodiments, the vinyl ester resin includes at least one of bisphenol A epoxy vinyl ester resin, phenolic epoxy vinyl ester resin, or addition polymerization epoxy vinyl ester resin with main chain structure, the curing agent includes methyl ethyl ketone peroxide, the thixotropic agent is polyamide wax, the thickener is bentonite, and the modified filler is surface activation modified copper powder. In some embodiments, the vinyl ester resin includes at least one of bisphenol A epoxy vinyl ester resin, phenolic epoxy vinyl ester resin, or addition polymerization epoxy vinyl ester resin with main chain structure, the curing agent includes methyl ethyl ketone peroxide, the thixotropic agent is polyamide wax, the thickener is bentonite, and the modified filler is surface activation modified nitrile rubber powder.

In some embodiments, the modified filler is prepared by preparing a processed filler and drying the processed filler. The processed filler is produced by soaking and stirring the filler in alcohol and a coupling agent. The coupling agent may include at least one of a silane coupling agent or a titanate coupling agent. Specifically, the filler may be soaked in the alcohol and the coupling agent, and stirred for a first time, and then the soaked filler may be evenly spread in an oven and dried at a first temperature to obtain the modified filler. In some embodiments, the first time is greater than 1 hour. In some embodiments, the first time is greater than 2 hours. In some embodiments, the first time is greater than 3 hours. In some embodiments, the first temperature is 50-80° C. In some embodiments, the first temperature is 55-75° C. In some embodiments, the first temperature is 60-70° C. In some embodiments, the drying time is at least 24 hours. In some embodiments, the drying time is at least 27 hours. In some embodiments, the drying time is at least 30 hours.

In some embodiments, the curing agent and at least one of the thixotropic agent, the thickener, or the modified filler are added to the vinyl ester resin together or separately. In some embodiments, the modified filler is added in multiple batches. In some embodiments, the modified filler is added while stirring. In some embodiments, the addition of the modified filler is completed within a second time. Merely by way of example, the second time is 10 min, 20 min, 30 min, or 1 h. By adding the modified filler multiple times and/or adding while stirring, the modified filler can be evenly dispersed in the vinyl ester resin. In some embodiments, all raw materials for preparing the acoustic matching material are added in a certain order. For example, the vinyl ester resin is added first, then the thixotropic agent, the curing agent, and the thickener are added in any order, and finally the modified filler is added. For example, the vinyl ester resin, the thixotropic agent, the curing agent, the thickener, and the modified filler are added in sequence. As another example, the vinyl ester resin, the curing agent, the thixotropic agent, the thickener, and the modified filler are added in sequence. As yet another example, the vinyl ester resin, the curing agent, the thickener, the thixotropic agent, and the modified filler are added in sequence.

In some embodiments, the vinyl ester resin added with the curing agent and at least one of the thixotropic agent, the thickener, or the modified filler is continuously stirred at a certain temperature (e.g., 25° C.) to react to form the mixture.

In preparation step 220, the mixture is cured to form the acoustic matching material.

In some embodiments, the mixture is poured into a mold, and then the mold is placed in an oven and cured at a second temperature for a third time to form the acoustic matching material. In some embodiments, the second temperature is 25-150° C. In some embodiments, the second temperature is 30-130° C. In some embodiments, the second temperature is 50-120° C. In some embodiments, the second temperature is 70-100° C. In some embodiments, the second temperature is 80-90° C. In some embodiments, the third time is 2-48 h. In some embodiments, the third time is 5-40 h. In some embodiments, the third time is 10-35 h. In some embodiments, the third time is 15-30 h. In some embodiments, the third time is 20-25 h.

The acoustic matching materials and methods for preparing thereof are described by examples below. It should be noted that the reaction conditions, the reaction materials, and amounts of reaction materials in the following examples are only for illustration and do not limit the scope of the present disclosure.

### Example 1

100 g vinyl ester resin was added to a 250 ml flask and stirred for 3 minutes at 25° C., and then 2 g curing agent (methyl ethyl ketone peroxide) was added to form a mixture. The mixture was mixed evenly and then poured into a mold. The mold containing the mixture was evacuated for 10 minutes, and then cured at room temperature for 48 hours to obtain an acoustic matching material.

Performance data of the acoustic matching material obtained in the Example 1 is as follows: flexural modulus is 3.82 GPa, density is 1.17 g/cm, acoustic impedance is 3.12 MRayl, sound attenuation at the frequency of 5 MHz is 6.3 dB/cm, and sound velocity is 2670 m/s.

### Example 2

100 g vinyl ester resin and 0.5 g thixotropic agent (polyamide wax) were added to a 250 ml flask and stirred for 3 minutes at 25° C., and then 2 g curing agent (methyl ethyl ketone peroxide) and 4 g thickener (bentonite) were added and stirred for 5 minutes until evenly dispersed, and then 400 g surface activation modified tungsten powder was added in four batches within 20 minutes to form a mixture. The mixture was mixed evenly and then poured into a mold. The mold containing the mixture was cured at room temperature for 48 hours to obtain an acoustic matching material.

Performance data of the acoustic matching material obtained in the Example 2 is as follows: flexural modulus is 4.45 GPa, density is 4.63 g/cm, acoustic impedance is 10.61 MRayl, sound attenuation at the frequency of 5 MHz is 22.5 dB/cm, and sound velocity is 2292 m/s.

### Example 3

100 g vinyl ester resin and 0.5 g thixotropic agent (polyamide wax) were added to a 250 ml flask and stirred for 3 minutes at 25° C., and then 2 g curing agent (methyl ethyl ketone peroxide) and 4 g thickener (bentonite) were added and stirred for 5 minutes until evenly dispersed, and then 12 g surface activation modified hollow glass microsphere was added in four batches within 20 minutes to form a mixture. The mixture was mixed evenly and then poured into a mold. The mold containing the mixture was cured at room temperature for 48 hours to obtain an acoustic matching material.

Performance data of the acoustic matching material obtained in the Example 3 is as follows: flexural modulus is 3.54 GPa, density is 0.74 g/cm, acoustic impedance is 1.82 MRayl, sound attenuation at the frequency of 5 MHz is 26.1 dB/cm, and sound velocity is 2455 m/s.

### Example 4

100 g vinyl ester resin and 0.5 g thixotropic agent (polyamide wax) were added to a 250 ml flask and stirred for 3 minutes at 25° C., and then 2 g curing agent (methyl ethyl ketone peroxide) and 4 g thickener (bentonite) were added and stirred for 5 minutes until evenly dispersed, and then 150 g surface activation modified aluminum oxide microsphere was added in four batches within 20 minutes to form a mixture. The mixture was mixed evenly and then poured into a mold. The mold containing the mixture was cured at room temperature for 48 hours to obtain an acoustic matching material.

Performance data of the acoustic matching material obtained in the Example 4 is as follows: flexural modulus is 4.72 GPa, density is 1.88 g/cm, acoustic impedance is 5.61 MRayl, sound attenuation at the frequency of 5 MHz is 23.8 dB/cm, and sound velocity is 2984 m/s.

### Example 5

100 g vinyl ester resin and 0.5 g thixotropic agent (polyamide wax) were added to a 250 ml flask and stirred for 3 minutes at 25° C., and then 2 g curing agent (methyl ethyl ketone peroxide) and 4 g thickener (bentonite) were added and stirred for 5 minutes until evenly dispersed, and then 100 g surface activation modified glass microsphere was added in four batches within 20 minutes to form a mixture. The mixture was mixed evenly and then poured into a mold. The mold containing the mixture was cured at room temperature for 48 hours to obtain an acoustic matching material.

Performance data of the acoustic matching material obtained in the Example 5 is as follows: flexural modulus is 4.48 GPa, density is 1.56 g/cm, acoustic impedance is 4.41 MRayl, sound attenuation at the frequency of 5 MHz is 24.3 dB/cm, and sound velocity is 2826 m/s.

### Example 6

100 g vinyl ester resin and 0.5 g thixotropic agent (polyamide wax) were added to a 250 ml flask and stirred for 3 minutes at 25° C., and then 2 g curing agent (methyl ethyl ketone peroxide) and 4 g thickener (bentonite) were added and stirred for 5 minutes until evenly dispersed, and then 200 g surface activation modified copper powder was added in four batches within 20 minutes to form a mixture. The mixture was mixed evenly and then poured into a mold. The mold containing the mixture was cured at room temperature for 48 hours to obtain an acoustic matching material.

Performance data of the acoustic matching material obtained in the Example 6 is as follows: flexural modulus is 4.1 GPa, density is 2.65 g/cm, acoustic impedance is 7.00 MRayl, sound attenuation at the frequency of 5 MHz is 19.5 dB/cm, and sound velocity is 2641 m/s.

### Example 7

100 g vinyl ester resin was added to a 250 ml flask and stirred for 3 minutes at 25° C., and then 2 g curing agent (methyl ethyl ketone peroxide) was added and stirred for 5 minutes until evenly dispersed, and then 30 g surface activation modified nitrile rubber powder was added in four batches within 20 minutes to form a mixture. The mixture was mixed evenly and then poured into a mold. The mold containing the mixture was cured at room temperature for 48 hours to obtain an acoustic matching material.

Performance data of the acoustic matching material obtained in the Example 7 is as follows: flexural modulus is 2.93 GPa, density is 1.12 g/cm, acoustic impedance is 2.85 MRayl, sound attenuation at the frequency of 5 MHz is 17.4 dB/cm, and sound velocity is 2549 m/s.

### Comparative Example 1

78 g epoxy resin and 24 g curing agent (methyl ethyl ketone peroxide) were added to a 250 ml flask and stirred for 5 minutes at 25° C. to form a mixture. The mixture was mixed evenly and then poured into a mold. The mold containing the mixture was evacuated for 10 minutes, and then cured at room temperature for 48 hours to obtain a material.

Performance data of the material obtained in the Comparative Example 1 is as follows: flexural modulus is 3.21 GPa, density is 1.15 g/cm, acoustic impedance is 3.14 MRayl, sound attenuation at the frequency of 5 MHz is 16.4 dB/cm, and sound velocity is 2733 m/s.

### Comparative Example 2

78 g epoxy resin and 0.5 g thixotropic agent (polyamide wax) were added to a 250 ml flask and stirred for 3 minutes at 25° C., and then 24 g curing agent (methyl ethyl ketone peroxide) and 4 g thickener (bentonite) were added and stirred for 5 minutes until evenly dispersed, and then 400 g surface activation modified tungsten powder was added in four batches within 20 minutes to form a mixture. The mixture was mixed evenly and then poured into a mold. The mold containing the mixture was cured at room temperature for 48 hours to obtain a material.

Performance data of the material obtained in the Comparative Example 2 is as follows: flexural modulus is 4.19 GPa, density is 4.41 g/cm, acoustic impedance is 10.22 MRayl, sound attenuation at the frequency of 5 MHz is 27.0 dB/cm, and sound velocity is 2317 m/s.

### Comparative Example 3

78 g epoxy resin and 0.5 g thixotropic agent (polyamide wax) were added to a 250 ml flask and stirred for 3 minutes at 25° C., and then 24 g curing agent (methyl ethyl ketone peroxide) and 4 g thickener (bentonite) were added and stirred for 5 minutes until evenly dispersed, and then 12 g surface activation modified hollow glass microsphere was added in four batches within 20 minutes to form a mixture. The mixture was mixed evenly and then poured into a mold. The mold containing the mixture was cured at room temperature for 48 hours to obtain a material.

Performance data of the material obtained in the Comparative Example 3 is as follows: flexural modulus is 3.15 GPa, density is 0.70 g/cm, acoustic impedance is 1.74 MRayl, sound attenuation at the frequency of 5 MHz is 35.7 dB/cm, and sound velocity is 2490 m/s.

The performance data of the acoustic matching materials obtained by the Examples 1-7 and the materials obtained by the Comparative Examples 1-3 are shown in Table 1 below:

TABLE 1
performance data of the acoustic matching materials obtained by the Examples
1-7 and the materials obtained by the Comparative Examples 1-3

| | | | Sound | | | |
|---|---|---|---|---|---|---|
| | | | attenuation | | | |
| | | | at the | | | |
| | | Sound | frequency | | Acoustic | Flexural |
| | | velocity | of 5 MHz | Density | impedance | modulus |
| Examples | Raw Materials | (m/s) | (dB/cm) | (g/cm3) | (MRayl) | (Gpa) |
| Comparative | 78 g epoxy resin + | 2733 | 16.4 | 1.15 | 3.14 | 3.21 |
| Example 1 | 24 g curing agent | | | | | |
| Comparative | 78 g epoxy resin + | 2317 | 27.0 | 4.41 | 10.22 | 4.19 |
| Example 2 | 0.5 g thixotropic | | | | | |
| | agent + 24 g curing | | | | | |
| | agent + 4 g thickener + | | | | | |
| | 400 g surface | | | | | |
| | activation modified | | | | | |
| | tungsten powder | | | | | |
| Comparative | 78 g epoxy resin + | 2490 | 35.7 | 0.70 | 1.74 | 3.15 |
| Example 3 | 0.5 g thixotropic | | | | | |
| | agent + 24 g curing | | | | | |
| | agent + 4 g thickener + | | | | | |
| | 12 g activation | | | | | |
| | modified hollow | | | | | |
| | glass microsphere | | | | | |
| Example 1 | 100 g vinyl ester | 2670 | 6.3 | 1.17 | 3.12 | 3.82 |
| | resin + 2 g curing | | | | | |
| | agent | | | | | |
| Example 2 | 100 g vinyl ester | 2292 | 22.5 | 4.63 | 10.61 | 4.45 |
| | resin + 0.5 g | | | | | |
| | thixotropic agent + | | | | | |
| | 2 g curing agent + 4 g | | | | | |
| | thickener + 400 g | | | | | |
| | surface activation | | | | | |
| | modified tungsten | | | | | |
| | powder | | | | | |
| Example 3 | 100 g vinyl ester | 2455 | 26.1 | 0.74 | 1.82 | 3.54 |
| | resin + 0.5 g | | | | | |
| | thixotropic agent + | | | | | |
| | 2 g curing agent + | | | | | |
| | 4 g thickener + 12 g | | | | | |
| | surface activation | | | | | |
| | modified hollow | | | | | |
| | glass microsphere | | | | | |
| Example 4 | 100 g vinyl ester | 2984 | 23.8 | 1.88 | 5.61 | 4.72 |
| | resin + 0.5 g | | | | | |
| | thixotropic agent + | | | | | |
| | 2 g curing agent + | | | | | |
| | 4 g thickener + 150 g | | | | | |
| | surface activation | | | | | |
| | modified aluminum | | | | | |
| | oxide microsphere | | | | | |
| Example 5 | 100 g vinyl ester | 2826 | 24.3 | 1.56 | 4.41 | 4.48 |
| | resin + 0.5 g | | | | | |
| | thixotropic agent + | | | | | |
| | 2 g curing agent + | | | | | |
| | 4 g thickener + 100 g | | | | | |
| | surface activation | | | | | |
| | modified glass | | | | | |
| | microsphere | | | | | |
| Example 6 | 100 g vinyl ester | 2641 | 19.5 | 2.65 | 7.00 | 4.10 |
| | resin + 0.5 g | | | | | |
| | thixotropic agent + | | | | | |
| | 2 g curing agent + | | | | | |
| | 4 g thickener + 200 g | | | | | |
| | surface activation | | | | | |
| | modified copper | | | | | |
| | powder | | | | | |
| Example 7 | 100 g vinyl ester | 2549 | 17.4 | 1.12 | 2.85 | 2.93 |
| | resin + 2 g curing | | | | | |
| | agent + 30 g surface | | | | | |
| | activation modified | | | | | |
| | nitrile rubber powder | | | | | |

Compared with the performance data of the material obtained in Comparative Example 1, the acoustic matching material obtained in the Example 1 has a higher flexural modulus, a slightly higher density, a slightly smaller acoustic impedance, and a smaller sound attenuation at the frequency of 5 MHz. Compared with the performance data of the materials obtained in Comparative Examples 1-3, the acoustic matching materials obtained in the Examples 1-3 have a higher flexural modulus. Therefore, the acoustic matching materials prepared in the embodiments of the present disclosure have higher mechanical strength and lower sound attenuation than the materials prepared using the epoxy resin as the matrix.

By comparing the performance data of the acoustic matching material obtained in Example 2 with the material obtained in Comparative Example 2, and comparing the performance data of the acoustic matching material obtained in Example 3 with the material obtained in Comparative Example 3, when the type and the addition ratio of the modified filler are the same (the modified filler in the Example 2 and Comparative Example 2 is 400 g surface activation modified tungsten powder, the modified filler in the Example 3 and Comparative Example 3 is 12 g surface activation modified hollow glass microsphere), the acoustic matching materials obtained in Example 2-3 have smaller sound attenuation (i.e., higher sound transmittance).

By comparing Examples 1-6, it can be seen that the acoustic matching materials with different acoustic impedances (e.g., in the range of 1.82-10.61 MRayl) are prepared by changing the type and the addition ratio of the modified filler, and the acoustic matching materials have a low sound attenuation (e.g., the sound attenuation at the frequency of 5 MHz is 6.3-26.1 dB/cm), which can be applied to different matching layers of the ultrasonic probe 110 and also meet the acoustic simulation requirements.

By comparing Examples 1 and 2-7, it can be seen that when the surface activation modified nitrile rubber powder is used as the modified filler, the prepared acoustic matching material has a smaller sound attenuation at the frequency of 5 MHz.

The technical effects of the embodiments of the present disclosure include, but are not limited to: (1) the acoustic matching material prepared in the embodiments of the present disclosure can replace the material prepared using epoxy resin as matrix and be applied to the at least one matching layer 112 of the ultrasonic probe 110; (2) compared with the materials prepared using epoxy resin as matrix, the acoustic matching materials prepared in the embodiments of the present disclosure have higher mechanical strength and lower sound attenuation; (3) the acoustic matching materials with different acoustic impedances are prepared by changing the type and the addition ratio of the modified filler, and the acoustic matching materials have a low sound attenuation; (4) compared with the materials prepared using epoxy resin as matrix, the acoustic matching materials prepared in the embodiments of the present disclosure cure faster, which can improve the production efficiency of the at least one matching layer 112; (5) compared with the materials prepared using epoxy resin as matrix, the acoustic matching materials prepared in the embodiments of the present disclosure have higher temperature resistance and greater flexural modulus, and accordingly have better molding and processing properties. It should be noted that different embodiments may produce different beneficial effects. In different embodiments, the possible beneficial effects may be any one or a combination of the above, or any other possible beneficial effects.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements, and amendments to the present disclosure. These modifications, improvements, and amendments are intended to be suggested by the present disclosure, and are within the spirit and scope of the exemplary embodiments of the present disclosure.

Meanwhile, the present disclosure uses specific words to describe the embodiments of the present disclosure. For example, “one embodiment”, “an embodiment”, and/or “some embodiments” refer to a certain feature, structure or characteristic related to at least one embodiment of the present disclosure. Therefore, it should be emphasized and noted that references to “one embodiment” or “an embodiment” or “an alternative embodiment” two or more times in different places in the present disclosure do not necessarily refer to the same embodiment. In addition, certain features, structures, or characteristics in one or more embodiments of the present disclosure may be properly combined.

In addition, unless clearly stated in the claims, the sequence of processing elements and sequences described in the present disclosure, the use of counts and letters, or the use of other names are not used to limit the sequence of processes and methods in the present disclosure. While the foregoing disclosure has discussed byway of various examples some embodiments of the invention that are presently believed to be useful, it should be understood that such detail is for illustrative purposes only and that the appended claims are not limited to the disclosed embodiments, but rather, the claims are intended to cover all modifications and equivalent combinations that fall within the spirit and scope of the embodiments of the present disclosure. For example, although the implementation of various components described above may be embodied in a hardware device, it may also be implemented as a software only solution, e.g., an installation on an existing server or mobile device.

In the same way, it should be noted that in order to simplify the expression disclosed in this disclosure and help the understanding of one or more embodiments of the invention, in the foregoing description of the embodiments of the present disclosure, sometimes multiple features are combined into one embodiment, drawings or descriptions thereof. This method of disclosure does not, however, imply that the subject matter of the disclosure requires more features than are recited in the claims. Rather, claimed subject matter may lie in less than all features of a single foregoing disclosed embodiment.

In some embodiments, counts describing the quantity of components and attributes are used. It should be understood that such counts used in the description of the embodiments use the modifiers “about”, “approximately” or “substantially” in some examples. Unless otherwise stated, “about”, “approximately” or “substantially” indicates that the stated figure allows for a variation of ±20%. Accordingly, in some embodiments, the numerical parameters used in the disclosure and claims are approximations that can vary depending upon the desired characteristics of individual embodiments. In some embodiments, numerical parameters should consider the specified significant digits and adopt the general digit retention method. Although the numerical ranges and parameters used in some embodiments of the present disclosure to confirm the breadth of the range are approximations, in specific embodiments, such numerical values are set as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. An acoustic matching material applied to a matching layer of an ultrasonic probe, wherein the acoustic matching material is prepared by:
adding additive into a matrix to form a mixture, the matrix including vinyl ester resin and a curing agent, the additive including at least one of a thixotropic agent, a thickener, or a modified filler; and
curing the mixture.

2. The acoustic matching material of claim 1, wherein at a frequency of 5 MHz, a sound attenuation of the acoustic matching material is less than 27 dB/cm.

3. The acoustic matching material of claim 2, wherein at the frequency of 5 MHz, the sound attenuation of the acoustic matching material is less than 16 dB/cm.

4. The acoustic matching material of claim 3, wherein at the frequency of 5 MHz, the sound attenuation of the acoustic matching material is less than 7 dB/cm.

5. The acoustic matching material of claim 1, wherein
a mass fraction of the vinyl ester resin is 100 parts,
a mass fraction of the curing agent is 1-2 parts,
a mass fraction of the thixotropic agent is 0-0.5 parts,
a mass fraction of the thickener is 0-4 parts, and/or
a mass fraction of the modified filler is 0-400 parts.

6. The acoustic matching material of claim 1, wherein a volume of the modified filler is 0-50% of a total volume of the mixture.

7. The acoustic matching material of claim 1, wherein the vinyl ester resin includes at least one of bisphenol A epoxy vinyl ester resin, phenolic epoxy vinyl ester resin, or addition polymerization epoxy vinyl ester resin with main chain structure, and the curing agent includes methyl ethyl ketone peroxide.

8. The acoustic matching material of claim 1, wherein the thixotropic agent includes at least one of hydrogenated castor oil or polyamide wax.

9. The acoustic matching material of claim 1, wherein the thickener includes at least one of bentonite or fumed silica.

10. The acoustic matching material of claim 1, wherein the modified filler is prepared by performing surface activation modification on a filler, the filler including at least one of hollow glass microsphere, glass microsphere, aluminum oxide microsphere, aluminum oxide powder, tungsten powder, tungsten trioxide powder, copper powder, iron powder, iron oxide powder, rubber powder, or plastic powder.

11. The acoustic matching material of claim 10, wherein the modified filler is prepared by:
preparing a processed filler by soaking and stirring the filler in alcohol and a coupling agent, the coupling agent includes at least one of a silane coupling agent or a titanate coupling agent; and
preparing the modified filler by drying the processed filler.

12. The acoustic matching material of claim 1, wherein acoustic matching materials with different acoustic impedances are prepared by changing a type and an addition ratio of the additive.

13. A method for preparing an acoustic matching material applied to a matching layer of an ultrasonic probe, comprising:
adding additive into a matrix to form a mixture, the matrix including vinyl ester resin and a curing agent, the additive including at least one of a thixotropic agent, a thickener, or a modified filler; and
curing the mixture.

14. The method of claim 13, wherein
a mass fraction of the vinyl ester resin is 100 parts,
a mass fraction of the curing agent is 1-2 parts,
a mass fraction of the thixotropic agent is 0-0.5 parts,
a mass fraction of the thickener is 0-4 parts, and/or
a mass fraction of the modified filler is 0-400 parts.

15. The method of claim 13, wherein the vinyl ester resin includes at least one of bisphenol A epoxy vinyl ester resin, phenolic epoxy vinyl ester resin, or addition polymerization epoxy vinyl ester resin with main chain structure, and the curing agent includes methyl ethyl ketone peroxide.

16. The method of claim 13, wherein
the thixotropic agent includes at least one of hydrogenated castor oil or polyamide wax, and/or
the thickener includes at least one of bentonite or fumed silica.

17. The method of claim 13, wherein the modified filler is prepared by performing surface activation modification on a filler, the filler including at least one of hollow glass microsphere, glass microsphere, aluminum oxide microsphere, aluminum oxide powder, tungsten powder, tungsten trioxide powder, copper powder, iron powder, iron oxide powder, rubber powder, or plastic powder.

18. The method of claim 17, wherein the modified filler is prepared by:
preparing a processed filler by soaking and stirring the filler in alcohol and a coupling agent, the coupling agent includes at least one of a silane coupling agent or a titanate coupling agent; and
preparing the modified filler by drying the processed filler.

19. An ultrasonic probe, comprising a matching layer that is made of an acoustic matching material, wherein the acoustic matching material is prepared by:
adding additive into a matrix to form a mixture, the matrix including vinyl ester resin and a curing agent, the additive including at least one of a thixotropic agent, a thickener, or a modified filler; and
curing the mixture.

20. The ultrasonic probe of claim 19, wherein different matching layers of the ultrasonic probe are made of acoustic matching materials with different acoustic impedances.
